(19) United States
(12) Patent Application Publication
(10) Pub. No.: US 2024/0233937 A1
(43) Pub. Date: Jul. 11, 2024

(54) INFORMATION PROCESSING SYSTEM AND INFORMATION PROCESSING METHOD

(71) Applicant: Johnson & Johnson Vision Care, Inc., Jacksonville, FL (US)

(72) Inventors: Tomotsugu Ohashi, Ascent (SG); Ryo Anzai, Tokyo (JP); Takuya Hanashima, Tokyo (JP); Naohisa Horikawa, Tokyo (JP); Hisashi Hanamure, Tokyo (JP); Norio Sasaki, Tokyo (JP)

(21) Appl. No.: 18/554,212

(22) PCT Filed: Apr. 7, 2022
(86) PCT No.: PCT/IB2022/053270

§ 371 Date: Oct. 6, 2023

(30) Foreign Application Priority Data
Apr. 7, 2021 (JP) 2021065170

(51) Int. Cl. G16H 40/67; G16H 50/20; G16H 50/30

(52) U.S. Cl. CPC G16H 40/67; G16H 50/20; G16H 50/30

(57) ABSTRACT

PROBLEM

To support maintaining health of monitored users.

RESOLUTION MEANS

The present invention includes:
determining means for determining the type of health information to monitor for a monitored user according to attributes of the monitored user,
acquiring means for acquiring monitored information of the monitored user corresponding to the determined type; and
notifying means to provide notification of acquired monitored user monitored information and/or information for monitoring health according to the determined type to the monitoring user associated with the monitored user.

### TECHNICAL FIELD

The present invention relates to an information processing system and an information processing method.

### CONVENTIONAL TECHNOLOGY

In recent years, services providing notification of the health status of monitored users such as working-age persons as well as children has been proposed. For example, Patent Document 1 describes an information processing device that provides notification of the health status of a plurality of subjects residing in a home, such as working-age persons or children or the like.

In recent years, there has been an increase in the number of elderly people living alone away from their families, and a service has been proposed to monitor the health of these elderly people and notify their families living separately. Patent Document 1 describes that if the server judges the health condition of the monitored user based on the detected information or the judged health condition meets prescribed conditions (for example, body temperature (epidermal temperature) is at or above a prescribed temperature (for example, 38° C.), stress level is at or above a prescribed value, or physical condition is judged to be poor), notification is provided to the user terminal of family living separately and thus notification can be provided for health conditions or difficulties of the monitored person.

### PRIOR ART DOCUMENTS

### Patent Document

Patent Document 1: Japanese Unexamined Patent Application Publication No. 2020-135132 A

### SUMMARY OF THE INVENTION

### Problem to Be Solved by the Invention

However, there is the problem that potential risks to health of the monitored user (unique based on user attributes) are not known to the user on the monitor side (hereinafter: monitoring user). Thus, the monitoring user is unable to make decisions regarding health information that should be monitored and/or information for monitoring health of the monitored user (for example, details of care when caring for the monitored user). Regarding this problem, Patent Document 1 does not include a concept of support from the management system side and this is a problem that has yet to be resolved.

In addition, with the technology of Patent Document 1, there is the problem that if the monitored user is not feeling well, notification is only provided to the user terminal of the family living separately. Even while not feeling well, the monitored user must submit an inquiry to a medical institution on their own and this is a heavy burden.

In addition, Patent Document 1 is limited to monitoring of general physical conditions such as body temperature (epidermal temperature). However, in the case that the monitored user is an elderly person, there is the problem that while an eye disease (for example, glaucoma, cataracts) unique to elderly persons may be present, the monitored user themselves may not be aware and family living separately also do not notice due to not having knowledge regarding eye diseases or difficulties unique to elderly persons. Similarly, if the monitored user is a child, even if there are symptoms of eye disease or difficulties (for example, squinting) unique to children, present, there is the problem that the child themselves is not aware and that family living in the same household may not be aware due to not having knowledge of eye diseases and difficulties unique to children. In this manner, there are problems that eye diseases (for example glaucoma, cataracts) or difficulties (for example, squinting) unique to a user's attributes, such as stage of life, that are present but the monitored user is not aware and where the monitoring user is also not aware due to not having knowledge of eye diseases or difficulties unique to the attributes of the monitored user.

In addition, in recent years the opportunities for modern persons (in particular, children) to come into contact with a digital device such as a smartphone or tablet has increased and so they are overused. This is causing the problem that the eye condition of modern persons (in particular, children) is difficult to maintain and deterioration of the eye condition of modern persons (in particular, children) needs to be prevented to the extent possible.

In light of the problems described above, an object of one aspect of the present invention is to provide an information processing system and information processing method that supports maintaining the health of a monitored user.

### Means for Solving the Problem

An information processing system of a first aspect of the present invention may include:
determining means for determining the type of monitored information to monitor for a monitored user according to user attributes of the monitored user; acquiring means for acquiring monitored information of the monitored user corresponding to the determined type; and
notifying means to provide notification of acquired monitored user monitored information and/or information for monitoring health according to the determined type to the monitoring user associated with the monitored user.

With this configuration, notification of the information type that should be monitored for the monitored user and/or information for monitoring health of the monitored user (for example care details for caring for the monitored user) is provided to the monitoring user. Therefore, the monitoring user can easily understand the information type that should be monitored for the monitored user and/or information for monitoring health of the monitored user (for example care details for caring for the monitored user).

With this information processing system, the notifying means may provide notification of the determined type to the monitored user and request this type of information of the monitored user and the acquiring means may acquire the information sent according to this request as monitored information.

With this configuration, the information processing system can acquire information needed to monitor the health condition of the monitored user and/or information for monitoring health and as a result, the monitoring user can understand health information of the monitored user.

An information processing system according to a second aspect of the present invention may include:
acquiring means for acquiring monitored information of the monitored user; judging means for judging a health condition of the monitored user using the acquired monitored information of the monitored user based on judgment criteria; and
notifying means to provide notification of acquired monitored user monitored information and/or information for monitoring health for the determined type based on the judgment results to the monitoring user associated with the monitored user.

With this configuration, if there is a difficulty with the health condition of the monitored user, notification of information of the monitored user and/or information for monitoring health of the determined type can be provided to the monitoring user enabling the monitoring user to quickly understand health difficulties of the monitored user.

The information processing system of this aspect may further include:
determining means for determining which type of monitored information to monitor for the monitored user according to user attributes of the monitored user, where
the acquiring means acquires monitored information of the monitored user corresponding to the determined type.

With this configuration, if there are any difficulties or symptoms of eye diseases (for example, glaucoma, cataracts) unique to the user's attributes such as stage of life, notification is provided to the monitoring user enabling reducing cases where the monitored user themselves are not aware and where family living together or separately are not aware due to not having knowledge regarding unique eye diseases or difficulties.

With the information processing system of this aspect, the judging means may determine judgment criteria according to user attributes of the monitored user.

With this configuration, health conditions of the monitored user can be judged using criteria that differs according to user attributes enabling understanding health conditions appropriately according to individual users. Note that examples of user attributes are described below.

The information processing system of this aspect where the notifying means may provide notification of at least one of:
information of the acquired monitored information of the monitored user formatted to enable submitting to a medical institution;
information enabling the monitoring user to communicate with a medical institution on behalf of the monitored user; or
information related to a diagnosis or treatment regarding health condition judged to be poor, abnormal, or to have deteriorated based on judgment criteria or regarding the medical institution,
as information including acquired monitored information of the monitored user and/or information for monitoring health of the determined type.

With this configuration, the monitoring user can use a monitoring user terminal to place an inquiry with a medical institution or receive medical consultation on behalf of the monitored user. Note that the information enabling communication of information by proxy with a medical institution typically includes the name and contact of the medical institution and information regarding the health condition and insurance of the monitored user needed for the inquiry. In addition, for cases where health conditions judged to be poor, abnormal, or deteriorating based on judgment criteria are discovered, illness information regarding the health conditions, information regarding care, and information regarding the medical institution to be consulted is provided. This enables reducing burden of submitting an inquiry to a medical institution if the monitored user is not feeling well.

The information processing system of this aspect, where the acquiring means acquires at least one of images and video of the eyes of the monitored user, eye movements obtained from the images and video of the eyes, and interaction data indicating behavior of work where eyes are used as monitored information, and the judging means judges the health condition of the eyes of the monitored user.

With this configuration, symptoms or signs of eye disease (for example, glaucoma, cataracts) or difficulties regarding the monitored user can be detected using a relatively simple method and notification can be provided to the monitoring user for performing a specialist test or diagnosis.

The information processing system of this aspect may further include:
an alert threshold determining means for determining time and/or frequency of viewing a digital device screen that is healthy according to user attributes of the monitored user and/or external factors as an alert threshold, where
the acquiring means acquires time and/or frequency of viewing a digital device screen by the monitored user as monitored information, and
the judging means judges whether or not the time and/or frequency of the monitored user viewing a digital device screen exceeds the threshold.

With this configuration, if the total time the monitored user is in contact with a digital device reaches an upper limit of time where contact with a digital device is healthy, notification can be provided to the monitoring user so worsening of eye conditions of the monitored user (in particular, children) can be prevented to some extent.

The information processing system of this aspect, where user attributes may be information expressing individual attributes of the monitored user and the monitoring user and that is information related to health and living environment including age, sex, vision, presence or absence of vision correction, chronic illness, medical history, surgical history, allergies, place of residence, living environment, living with other family members, and lifestyle.

With this configuration, the health condition of the monitored user can be understood and information to be monitored and information type where notification is to be provided to the monitoring user can be determined and associated with the user attributes of the user. Note that as long as the information related to the nature of the user satisfies this application, the information can be used as a user attribute.

The information processing system of this aspect, where the notifying means may provide notification of information including acquired monitored information of the monitored user and/or information for monitoring health of the determined type, or information including information related to user attributes of the monitored user or information related to user attributes of the monitoring user.

With this configuration, there is no relation with the user attributes of the monitoring user enabling providing notification of information regarding health risk or care of the monitored user even if the monitoring user has no knowledge or interest in the information and thus knowledge enabling appropriate care can be conveyed.

### Effect of the Invention

The first aspect of the present invention achieves:
(1) simplifies understanding by the monitoring user regarding type of health information to be monitored for the monitored user and/or information for monitoring health of the monitored user,
(2) reduces burden on the monitored user of submitting an inquiry to a medical institution when they are not feeling well,
(3) reduces cases where the monitored user is not aware of symptoms of eye disease (for example, glaucoma, cataracts) or difficulties unique to user's attributes such as stage of life and family living together or separately are also not aware due to not having knowledge regarding unique eye disease or difficulty, and
(4) prevents to a certain degree worsening of eye conditions of the monitored user (for example, children).

### BRIEF DESCRIPTION OF THE DRAWINGS

FIG. 1 is a schematic diagram of an information processing system according to the present embodiment.

FIG. 2 is a schematic diagram of a terminal for a monitored user's terminal according to the present embodiment.

FIG. 3 is a schematic diagram of an computer system according to the present embodiment.

FIG. 4 is a sequence diagram of a first example of processing according to the present embodiment

FIG. 5 is a sequence diagram of a second example of processing according to the present embodiment

FIG. 6 is a sequence diagram of a third example of processing according to the present embodiment

FIG. 7 is a sequence diagram of a fourth example of processing according to the present embodiment

### DETAILED DESCRIPTION OF THE INVENTION

Each embodiment will be described below with reference to the drawings. However, detailed description may be omitted more than necessary. For example, detailed descriptions on already well-known matters and redundant descriptions on substantially identical configurations may be omitted. This is to avoid unnecessarily redundancy in the following description and to facilitate the understanding of those skilled in the art.

The present embodiment provides means to solve the following issues.
(1) Simplifies understanding by the monitoring user regarding type of health information of the monitored user to monitor and/or understand information regarding managing the health of the monitored user.
(2) Enables reducing burden of submitting an inquiry to a medical institution if the monitored user is not feeling well.
(3) Reduces the number of cases where eye diseases (for example glaucoma, cataracts) or difficulties (for example, squinting) unique to a user's attributes, such as stage of life, are present, but the monitored user is not aware and where the monitoring user is also not aware due to not having knowledge of eye diseases or difficulties unique to the monitored user.
(4) Prevents the deterioration of eye conditions of the monitored user (for example, children) to the extent possible.

The monitored information according to the present embodiment is information regarding the monitored user for monitoring. In addition to information regarding health of the monitored user that is health information, this includes eye movements obtained from images/video of the monitored user, interaction data of actions where eyes are used, as well as amount of time and/or frequency the monitored user views a digital device screen.

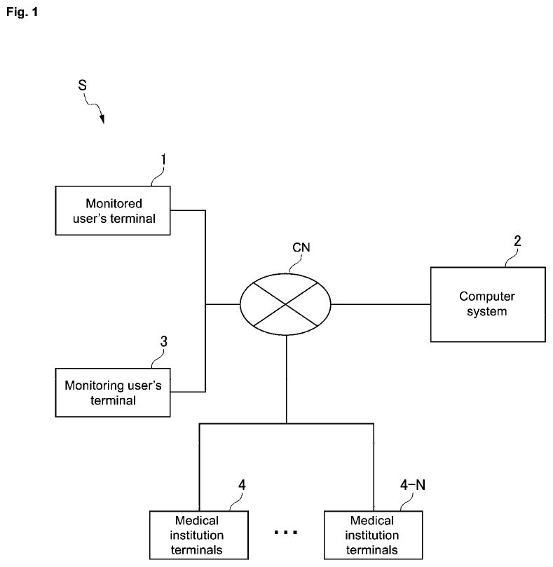

FIG. 1 is a schematic diagram of an information processing system according to the present embodiment. As shown in FIG. 1, an information processing system S includes a monitored user's terminal 1, a computer system 2 connected to this monitored user's terminal 1 via a communication network CN, a monitoring user's terminal 3 connected to this computer system 2 via the communication network CN, and medical institution terminals 4-1 to 4-N (where N is a natural number).

The monitored user's terminal 1 is a terminal used by the monitored user, for example, a cell phone such as a multifunctional cell phone (so-called smart phone), a tablet, a notebook computer, a desktop computer, or the like. In the present embodiment, the monitored user's terminal 1 is assumed to be able to exchange information with the computer system 2, for example, via a web browser.

Note, information exchange is not limited to a web browser for the computer system 2 and a specific application can be downloaded and installed on the monitored user's terminal 1 and information exchanged with the computer system 2 via this specific application.

The computer system 2 is used or managed by a management organization that manages the information processing system S according to the present embodiment. The computer system 2 provides information to the monitored user's terminal 1, the monitoring user's terminal 3, and the medical institution terminals 4-1 to 4-N. The computer system 2 may be a single computer or a plurality of computers.

The monitoring user's terminal 3 is a terminal used by the monitoring user who monitors the monitored user, and can be for example, a cell phone such as a multi-functional cell phone (so-called smartphone), a tablet, a notebook computer, a desktop computer, or the like. Here, the monitoring user is a user who has received approval from the monitored user (for example, a friend of the monitored user) or is a relative of the monitored user (relatives include guardians and family). In the present embodiment, the monitoring user's terminal 3 is assumed to be able to exchange information with the computer system 2, for example, via a web browser.

Note, information exchange is not limited to a web browser for the computer system 2 and a specific application can be downloaded and installed on the monitoring user's terminal 3 and information exchanged with the computer system 2 via this specific application.

Medical institution terminals 4-1 to 4-N are terminals used by employees (for example, doctors, nurses) of medical institutions and are, for example, cell phones such as multifunctional mobile phones (so-called smart phones), tablets, notebook computers, or desktop computers. In the present embodiment, the medical institution terminals 4-1 to 4-N are assumed to be able to exchange information with the computer system 2, for example, via a web browser.

Note, information exchange is not limited to a web browser for the computer system 2 and a specific application can be downloaded and installed on the medical institution terminals 4-1 to 4-N and information exchanged with the computer system 2 via this specific application. Hereinafter, medical institution terminals 4-1 to 4-N can be collectively referred to as medical institution terminal 4.

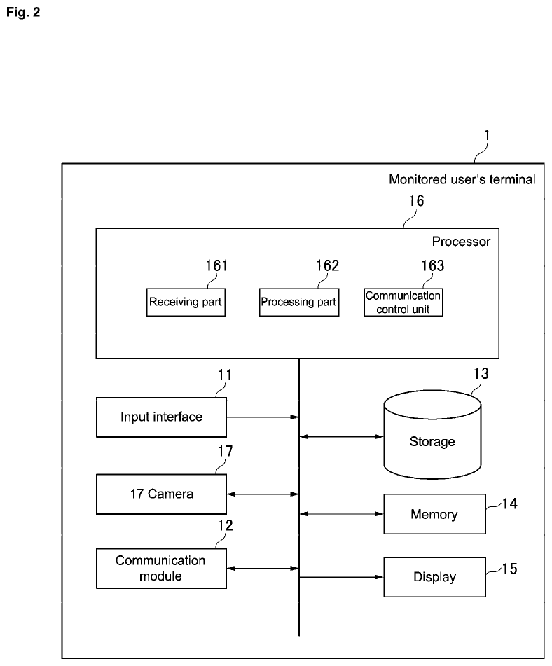

FIG. 2 is a schematic diagram of a monitored user's terminal according to the present embodiment. As illustrated in FIG. 2, the monitored user's terminal 1 includes, for example, an input interface 11, a communication module 12, storage 13, memory 14, display 15, a processor 16, and a camera 17.

The input interface 11 accepts input from the user and outputs an input signal corresponding to the accepted input to the processor 16.
The communication module 12 is connected to the communication network CN and communicates with the computer system 2 connected to the communication network CN. This communication can be wired or wireless, but is assumed to be wireless.

The storage 13 stores programs and various data read and executed by the processor 16.

The memory 14 temporarily holds data and programs. The memory 14 is a volatile memory, for example a random access memory (RAM).

The display 15 displays information using video signals that are input in accordance with commands of the processor 16. The display 15 is not limited to being built into the monitored user's terminal 1, but may also be connected to the monitored user's terminal 1 externally.

The processor 16 loads a program according to the present embodiment from storage 13 into memory 14 and by executing the series of commands included in this program, functions as a receiving part 161 that receives operations by a user, a processing part 162 that executes processing, and a communication controller 163 that controls a communication module.
The camera 17 captures images according to commands from the processor 16. The camera 17 is, as an example, a built-in camera of a smartphone, and is capable of capturing video of the eyes of the monitored user. The image/video obtained from the image capture is stored in storage 13. As a result, the image/video of the eyes of the monitored user is stored in the storage 13.

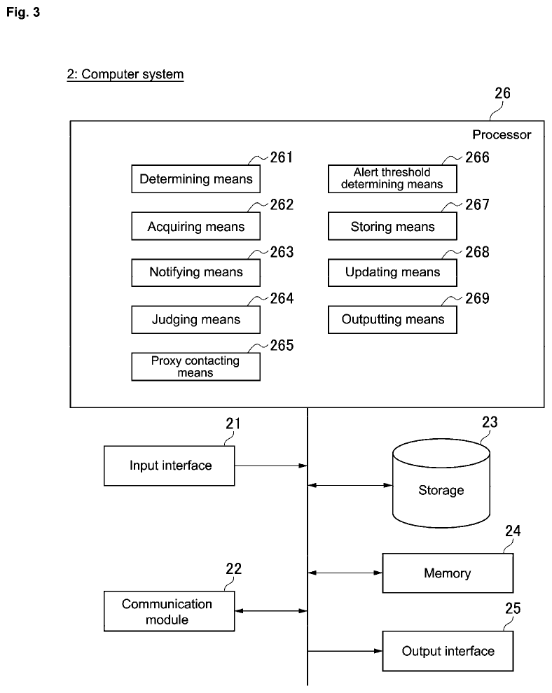

FIG. 3 is a schematic diagram of a computer system according to the present embodiment. As illustrated in FIG. 3, the computer system 2 includes an input interface 21, a communication module 22, storage 23, memory 24, an output interface 25, and a processor 26.

The iput interface 21 receives input from an administrator of the computer system 2 (for example, an employee of the management organization), and outputs an input signal corresponding to the received input to the processor 26.
The communication module 22 is connected to the communication network CN, and communicates with the monitored user's terminal 1, the monitoring user terminal, and the medical institution terminals 4-1 to 4-N. This communication can be wired or wireless, but is assumed to be wired.

The storage 23 stores programs and various data read and executed by the processor 26.

The memory 24 temporarily holds data and programs. The memory 24 is volatile memory, for example random access memory (RAM).
The output interface 25 can be connected to an external device and can output signals to this external device.

The processor 26 loads a program from storage 23 into the memory 24 and executes a series of commands included in the program and as a result functions as determining means 261, acquiring means 262, notifying means 263, judging means 264, proxy contacting means 265, alert threshold determining means 266, storing means 267, updating means 268, and outputting means 269. Each of the processes will be described later.

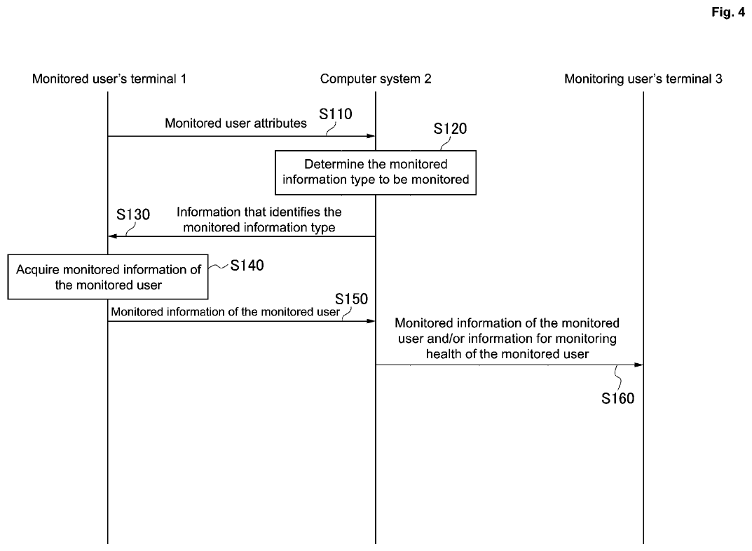

FIG. 4 is a sequence diagram of a first example of processing according to the present embodiment.

(Step S110) First, the monitored user's terminal 1 sends the attributes of the monitored user to the computer system 2.

(Step S120) Next, the determining means 261 of the computer system 2 determines which of the monitored information types to monitor for the monitored user based on the attributes of the monitored user. In this case, for example, the user attributes (for example, stage of life) are stored in storage 23 associated with types of monitored information to be monitored, and the determining means 261 may refer to storage 23 and make a determination corresponding to the user attributes of the monitored user. For example, if the monitored user's stage of life is sixties or older, the determining means 261 may determine “glaucoma” and/or “cataract” for “persons at stage of life of sixties or older” as the type of monitored information to be monitored.

(Step S130) Next, the computer system 2 outputting means 269 sends the information identifying the determined (health information) type to the monitored user's terminal 1.

(Step S140) Next, the monitored user's terminal 1 acquires the information to be monitored that is the type determined (for example, health information) from the monitored user. For example, if the determined type is “glaucoma and/or cataracts,” the health information is the presence, condition, or progression of the monitored user's “glaucoma and/or cataracts” and can be acquired by receiving input of the monitored user into the monitored user's terminal 1.

(Step S150) The monitored user's terminal 1 sends the acquired monitored information of the monitored user (for example health information) to the computer system 2.

(Step S160) Next, the acquiring means 262 of the computer system 2 acquires the monitored information of the monitored user. Furthermore, the notifying means 263 of the computer system 2 provides monitored information of the monitored user of the determined type and/or information for monitoring health corresponding to the determined type to the monitoring user. Here, information related to type and information for monitoring health are stored in the storage 23 in an associated manner. Therefore, the notifying means 263 may access the storage 23 and read information for monitoring health corresponding to the determined type to provide notification. For example, if the determined type is “glaucoma and/or cataracts,” health information that is an example of monitored information is presence, condition, or progression of “glaucoma and/or cataracts” of the monitored user and information for health monitoring is information regarding “glaucoma and/or cataracts” (for example, details regarding glaucoma and/or cataract care, treatment methods, cases, basic knowledge and the like).

In this manner, the computer system 2 of the present embodiment includes:
determining means 261 that determines what monitored information type to monitor regarding the monitored user according to user attributes of the monitored user,
acquiring means 262 that acquires monitored information corresponding to the determined type (for example, health information),
and notifying means 263 that provides notification of the monitored information of the monitored user of the determined type (for example, health information) and/or information for the health monitoring of the determined type to the monitoring user who monitors the monitored user.

With this configuration, notification of the monitored information type that should be monitored for the monitored user and/or information for monitoring health of the monitored user (for example care details for caring for the monitored user) is provided to the monitoring user. Therefore, the monitoring user can easily understand the monitored information type that should be monitored for the monitored user and/or information for monitoring health of the monitored user (for example care details for caring for the monitored user).

Typically, user attributes include, but are not limited to, the monitored user and monitoring user's personal attributes such as age, stage of life, and gender; health-related information such as eye condition (for example, visual acuity), presence or absence of vision correction, chronic illness, medical history, surgical history, and presence or absence of allergies; living environment information such as place of residence, living environment, presence or absence of co-residence with other family members, and living habits; and life events (for example, schooling, marriage) but are not limited to these. The user's attributes are used in the present embodiment, for example, as a decision-making tool for determining the type of health information to be monitored and the type of health management information to be notified for the monitored user. Any information about the nature of the user that satisfies this purpose can be treated as a user attribute. User attributes for a particular user can be changed as appropriate in accordance with life events (such as growth, moving, marriage, contracting a disease or the like) of the user over time.

These types of user attributes of the monitored user or monitoring user are preferably stored in storage 23 but if in a form accessible by the computer system 2 (for example, determining means 261), they may be stored on some other type of storing medium. For example, user attributes may, in general, be stored on each of the user terminals (for example, monitored user's terminal 1 or monitoring user's terminal 3) storage (for example, storage 13) or cloud storage area (not shown) for each user. Note, the determining means 261 may estimate other types of user attributes based on the user attribute referenced. For example, if a user's birthday is referenced, the user's age may be estimated from a comparison with the current date.

### Examples of the Types of Monitored Information

Monitored information is information related to the monitored user and indicates various types of information for directly or indirectly determining or estimating the health status of the monitored user in the computer system 2. Here, a specific example of a type of monitored information to be monitored will be described. Types of monitored information of the monitored user include, for example, health information that is information regarding the health of the user as well as information regarding wearing of contact lenses, purchase history of contact lenses, location information of the monitored user, number of steps, time and/or frequency of viewing a terminal screen, and distance from the user's eyes to the terminal screen. Here, health information includes input health diagnosis results, diagnosis information from a doctor (clinical record information), prescription information (for example, contact lens treatment information), or monitor information from the monitored user's terminal (or external) camera or sensor, for example, image or video capture of eyes, fundus photographs, view angle, focal distance, intraocular pressure, axial length, refractive power, a questionnaire or results of a simple eye exam, and other health information related to the eyes.

The type of monitored information to be monitored for an individual monitored user may be set or changed according to the user attributes of the monitored user. Specifically, the determining means 261 refers to the user attributes of the monitored user and determines the monitored information type to be monitored according to the user attributes. This decision can be performed by referring to a lookup table (not shown) for user attributes and what monitored information type to be monitored stored in storage 23, using information obtained externally such as the internet, or can be performed based on analysis results of similar past data.

The notifying means 263 can provide notification of the monitored information type set to be monitored to the monitored user and/or the monitoring user, and can request this type of monitored information (for example, health information) from the monitored user. In accordance with this request, the monitored user executes an operation (for example, camera operation) and/or inputs this type of monitored information (for example, contact lens information or contact lens purchase history). In this case, the acquiring means 262 may acquire the monitored information from the information sent for this request.

For example, if the monitored user is an elderly person, there is risk of eye diseases unique to elderly persons (for example, cataracts) so images of eyes are preferably monitored daily. On the other hand, there is also the possibility that both the monitored user and monitoring user are not aware of the risk and are not monitoring eye images. Here, the computer system 2 confirms user attributes of the monitored user and if there is risk of eye disease (for example, cataracts) unique to elderly persons above a certain age, requests photographing and sending eye images for monitoring health status related to eye disease. This request can be information for educating the user regarding monitoring or can be to perform operation promoting booting up a photo capture application of the monitored user's terminal 1 processor 16 and capturing an image along with a prescribed message.

### Specific Example of Monitored User Health Information Provided to the Monitoring User

Next, a specific example of providing notification of monitored user health information to the monitoring user will be described. A specific example of health information of the monitored user being provided to the monitoring user is as described below.
(1) Monitored user health information received as input or information from monitoring the monitored user (for example, results of a medical diagnosis, use of a smartphone by family that is remote, or the like)
(2) Information indicating health condition from analysis of input and received/monitored information based on user attributes (for example, cataract diagnostic results from artificial intelligence (AI) analysis of eye images that were taken, analysis report of health condition based on number of steps and screen time)
(3) Information acquired externally based on input and received/monitored information (for example, information on nearby specialist hospitals or pollen information for patients with hay fever)
(4) Information related to user attributes of the monitored user that are not related to user attributes of the monitoring user

Here, the (4) information related to user attributes of the monitored user that are not related to user attributes of the monitoring user anticipates a case where, for example, the monitored user is a senior citizen while the monitoring user is a child who has reached adulthood (otherwise, a guardian). Information (diagnostic information such as for cataracts or presbyopia or specialist hospital information) related to user attributes of senior citizens (age and medical history) may be information not related to user attributes of the monitoring user. Here, the monitoring user may not realize the health risk of cataracts or presbyopia and therefore may not be aware that the monitored user may be at risk, of the need for care such as diagnosis, or of information needed for care (type of risks, specialist hospitals, and lifestyle related improvements, and the like). Herein, even if there is no relation to user attributes of the monitoring user, the computer system 2 can automatically provide notification of information regarding health of the monitored user so even if there is a health risk that the monitoring user is not aware of, knowledge enabling appropriate care can be conveyed.

### Specific Example Related to Information for Stage of Life and Monitoring Health

A specific example regarding information for health management provided to the monitored user based on stage of life of the monitored user is described below. For example, if the monitored user is a teenager, the information for health management is “progress and suppression of nearsightedness,” “information for starting use of contact lenses,” “difference between near sightedness, far sightedness, and astigmatism,” “sports and contacts,” “relationship between eyes and ultraviolet light,” “relationship and countermeasures for contact lenses and hay fever,” “hyperemia while wearing contact lenses,” “information on smartphone presbyopia,” “safety of color contact lenses,” “relationship between contact lenses and conjunctivitis,” “information about a sty,” and the like.

For example, if the monitored user is in their twenties, information for monitoring health is “relationship between dry eyes and contact lens,” “risks of wearing contact lens,” and the like, in addition to that described above.
For example, if the monitored user is in their thirties, information for monitoring health is “information related to eyelid twitching” and can include “difference of nearsightedness, farsightedness, and astigmatism,” “suppression of progression of nearsightedness,” “relationship between contact lenses and conjunctivitis,” and “information related to sties” for those with children 0 to 9 years old in addition to that described above.
For example, if the monitored user is in their sixties or older, information for monitoring health may be “information related to presbyopia,” “information related to glaucoma,” and “information related to cataracts” in addition to that described above.

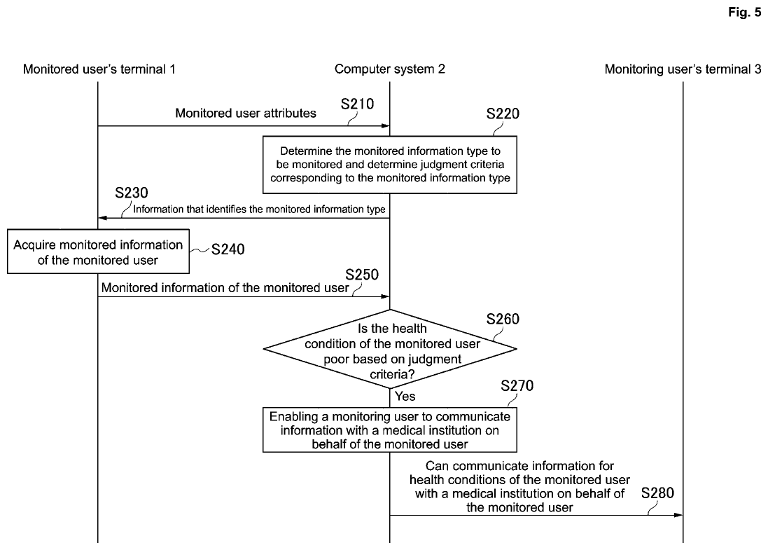

FIG. 5 is a sequence diagram of a second example of processing according to the present embodiment.

(Step S210) First, the monitored user's terminal 1 sends the attributes of the monitored user to the computer system 2.

(Step S220) Next, the determining means 261 of the computer system 2 determines which of the monitored information types to monitor for the monitored user based on the attributes of the monitored user. For example, if the monitored user's stage of life is sixties or older, the determining means 261 determines “glaucoma” and/or “cataract” as the type of monitored information to be monitored. Furthermore, the determining means 261 of the computer system 2 determines judgment criteria corresponding to this type of health information. For example, if the monitored information type is “glaucoma and/or cataracts”, judgment criteria corresponding to “glaucoma and/or cataracts” is set.

(Step S230) Next, the computer system 2 outputting means 269 sends the information identifying the determined (monitored information) type to the monitored user's terminal 1.

(Step S240) Next, the monitored user's terminal 1 acquires the monitored information that is the type determined from the monitored user. For example, if the determined type is “glaucoma and/or cataracts,” monitored information is images/video of the monitored user's eyes and these are used to judge presence of “glaucoma and/or cataracts” of the monitored user as well as status or progression.

(Step S250) The monitored user's terminal 1 sends the acquired monitored information of the monitored user to the computer system 2.

(Step S260) Next, the judging means 264 of the computer system 2 uses the monitored information (for example, images/video of the monitored user's eyes) and judges whether or not the health condition (for example, eye condition) of the monitored user is poor based on judgment criteria determined in step S220. Note, judgment regarding whether or not there is a difficulty can be made based on the judgment criteria or whether or not health condition has deteriorated based on judgment criteria.

(Step S270) If the health condition of the monitored user is judged to be poor (or abnormal or deteriorating) based on judgment criteria in step S260, the monitoring user can use the proxy contacting means 265 to communicate with a medical institution on behalf of the monitored user.

(Step S280) Furthermore, the notifying means 263 transmits the fact that information communication of health conditions of the monitored user with a medical institution on behalf of the monitored user is feasible to the monitoring user's terminal 3. Thus, the monitoring user can use a monitoring user's terminal 3 to place an inquiry with a medical institution or receive medical consultation on behalf of the monitored user. This enables reducing burden of submitting an inquiry to a medical institution if the monitored user is not feeling well. Here, based on the judgment results, notifying means 263 may provide notification of acquired monitored user health information and/or information for monitoring health for the determined type to the monitoring user associated with the monitored user. Specifically, the notifying means 263 may provide notification of at least one of:
information of the acquired health information of the monitored user formatted to enable submitting to a medical institution;
information enabling the monitoring user to communicate with a medical institution on behalf of the monitored user; or
information related to a diagnosis or treatment regarding health condition judged to be poor, abnormal, or to have deteriorated based on judgment criteria or to the medical institution,
as information including acquired health information of the monitored user and/or information for monitoring health of the determined type.

With this configuration, the monitoring user can use a monitoring user's terminal to place an inquiry with a medical institution or receive medical consultation on behalf of the monitored user. Note that the information enabling communication of information by proxy with a medical institution typically includes the name and contact of the medical institution and information regarding the health condition and insurance of the monitored user needed for the inquiry. In addition, for cases where health conditions judged to be poor, abnormal, or deteriorating based on judgment criteria are discovered, illness information regarding the health conditions, information regarding care, and information regarding the medical institution to be consulted is provided. This enables reducing burden of submitting an inquiry to a medical institution if the monitored user is not feeling well.

In this manner, the computer system 2 includes:
acquiring means 262 for acquiring health information from the monitored user;
judging means 264 that judges the health condition of the monitored user using the health information of the monitored user; and
in case the health information of the monitored user is judged to be worse than the judgment criteria, a proxy contacting means 265 enabling the monitoring user to communicate information with a medical institution on behalf of the monitored user.

With this configuration, the monitoring user can use a monitoring user's terminal 3 to place an inquiry with a medical institution or receive medical consultation on behalf of the monitored user. This enables reducing burden of submitting an inquiry to a medical institution if the monitored user is not feeling well.

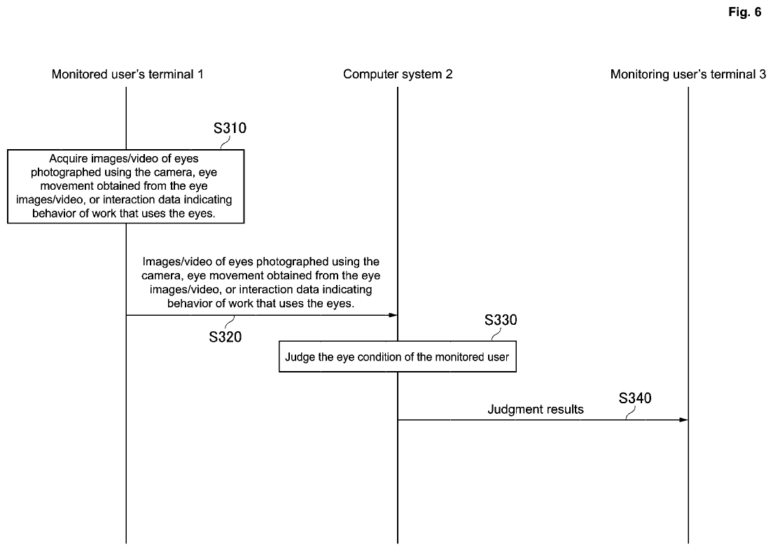

FIG. 6 is a sequence diagram of a third example of processing according to the present embodiment

(Step S310) First, the monitored user's terminal 1 acquires eye images/video photographed using the camera 17, eye movement obtained from the eye images/video, or interaction data indicating behavior for work that uses the eyes.

(Step S320) Next, the monitored user's terminal 1 sends this information (in other words, eye images/video photographed using the camera 17, eye movement obtained from the eye images/video, or interaction data indicating behavior for work that uses the eyes) to the computer system 2.

(Step S330) Next, judging means 264 of the computer system 2 receives this information and judges eye conditions of the monitored user based on this information (in other words, eye images/video photographed using the camera 17, eye movement obtained from the eye images/video, or interaction data indicating behavior for work that uses the eyes).

For example, the judging means 264 can judge presence or absence of glaucoma and/or cataracts and condition or progression from these images/video of the monitored user's eyes. This judgment can be performed by judgment comparing features of the images/video of the user with criteria features or by judgment by a trained machine learning model.

(Step S340) The notifying means 263 of the computer system 2 notifies the monitoring user of the judgment results. Here, as an example, the notifying means 263 sends the judgment results to the monitoring user's terminal 3.

Thus, the computer system 2 according to the present embodiment includes:
acquiring means 262 that acquires eye images/video photographed using the camera 17, eye movement obtained from the eye images/video, or interaction data indicating behavior for work that uses the eyes from the monitored user;
judging means 264 that judges the eye condition of the monitored user using the eye images/video photographed using the camera 17, eye movement obtained from the eye images/video, or interaction data indicating behavior for work that uses the eyes; and
notifying means 263 that provides notification of the judgment results to the monitoring user who monitors the monitored user.

With this configuration, if there are any difficulties or symptoms of eye diseases (for example, glaucoma, cataracts) unique to the user's attributes such as stage of life, notification is provided to the monitoring user enabling reducing cases where the monitored user themselves are not aware and where family living together or separately are not aware due to not having knowledge regarding unique eye diseases or difficulties.

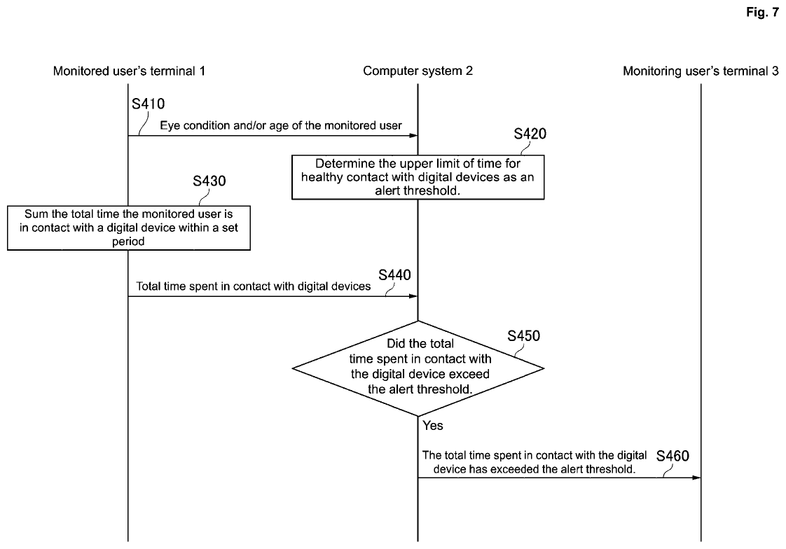

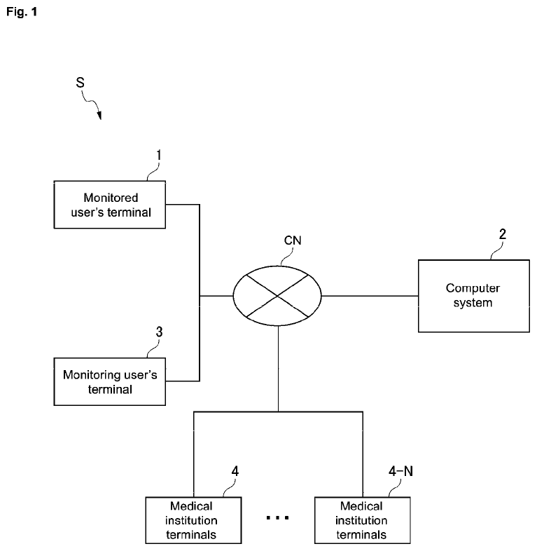

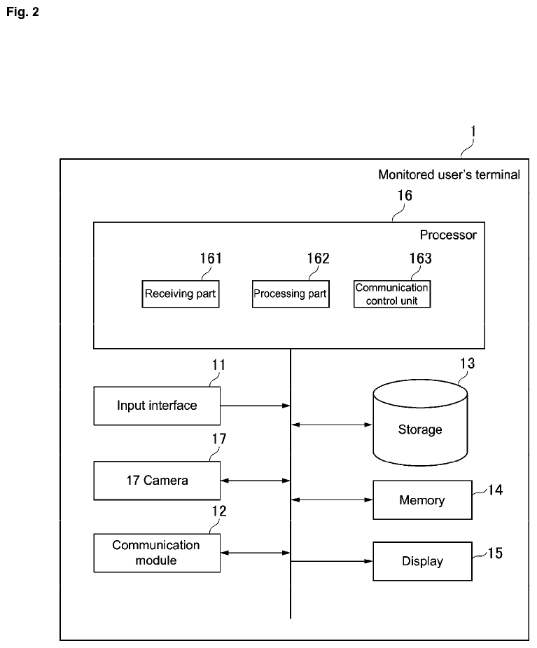

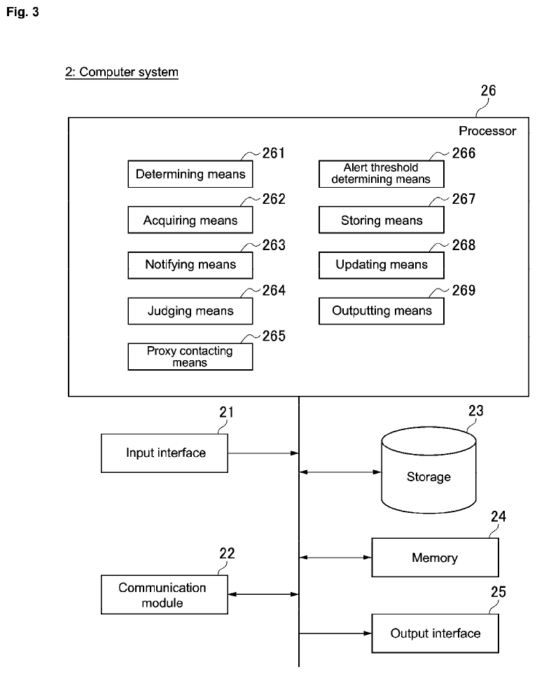

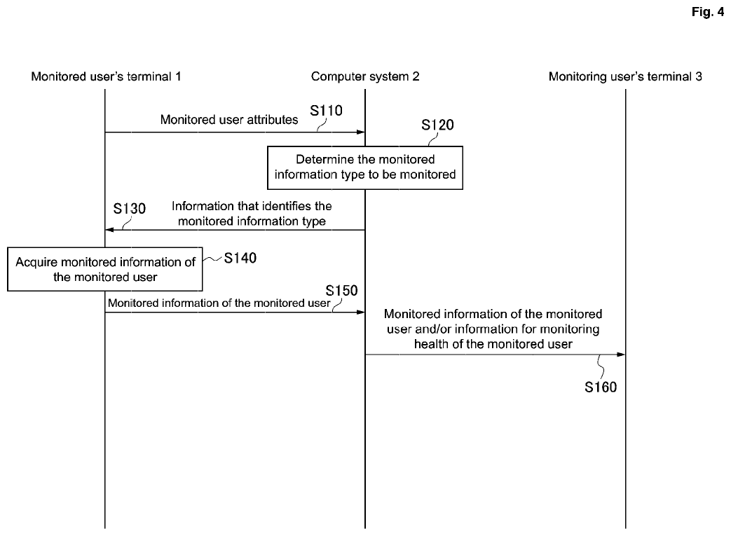

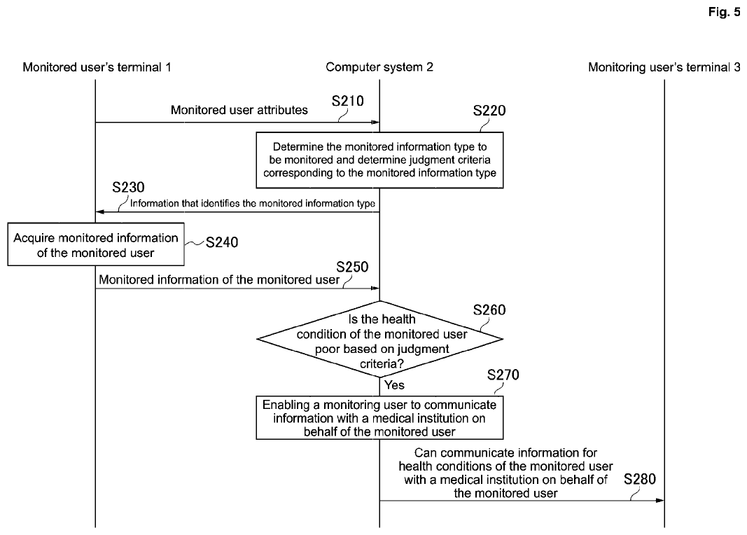

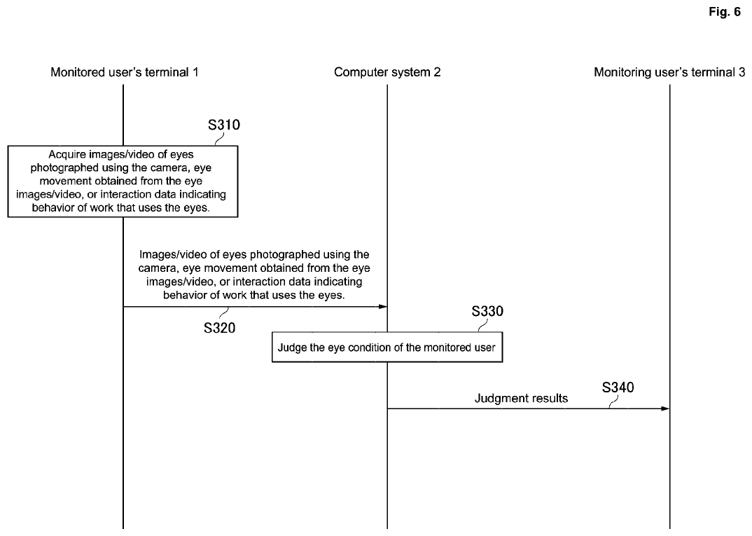

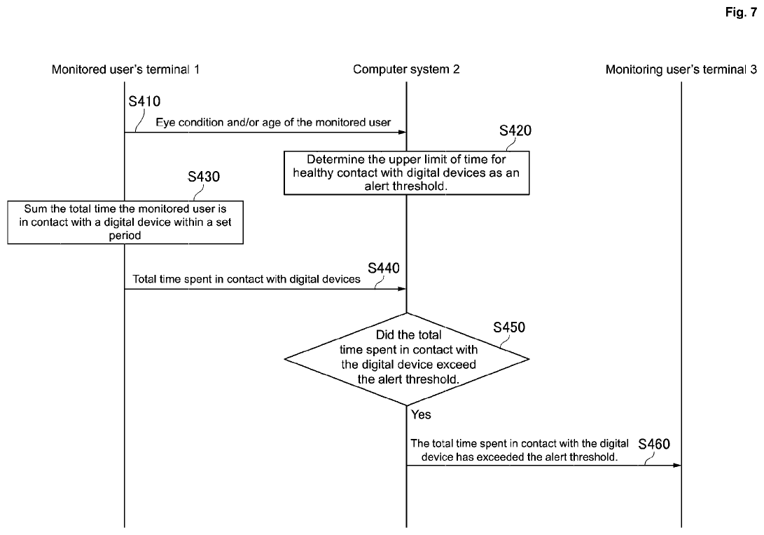

FIG. 7 is a sequence diagram of a fourth example of processing according to the present embodiment

(Step S410) First, the monitored user's terminal 1 sends the eye condition and/or age of the monitored user to the computer system 2.

(Step S420) The alert threshold determining means 266 of the computer system 2 sets an upper limit on time where contact with a digital device is healthy according to eye conditions and/or age of the monitored user and/or external factors (for example weather, pollen dispersal, amount of UV radiation, season, and the like).

(Step S430) Next, the monitored user's terminal 1 sums the total time the monitored user is in contact with a digital device within a set period.

(Step S440) Next, the monitored user's terminal 1 sends the total time that the monitored user is in contact with a digital device within a set period obtained through summation to the computer system 2.

(Step S450) Next, the judging means 264 of the computer system 2 judges whether or not the total time that the monitored user was in contact with the digital device within the set period exceeded the alert threshold.

(Step S460) If the result of the judgment by the judging means 264 is that the total time exceeds the alert threshold, the notifying means 263 provides notification to the monitoring user. As an example, the notifying means 263 sends the fact that the total amount of time in contact with a digital device exceeded the alert threshold to the monitoring user's terminal 3.

In this manner, the computer system 2 according to the present embodiment includes:
acquiring means 262 that acquires total time the monitored user is in contact with a digital device within a set period and eye conditions and/or age of the monitored user;
alert threshold determining means 266 that determines an upper limit of time that is healthy for contact with a digital device according to eye condition and/or age and/or external factors of the monitored user as an alert threshold;
judging means 264 that judges whether or not the total time that the monitored user has been in contact with a digital device within a set period exceeds the alert threshold; and
notifying means 263 that provides notification to the monitoring user if the judgment results from the judging means 264 are that the total time exceeds the alert threshold.

With this configuration, if the total time the monitored user is in contact with a digital device reaches an upper limit of time of contact with a digital device being healthy, notification can be provided to the monitoring user so worsening of eye conditions of the monitored user (in particular, children) can be prevented to some extent.

### Another Embodiment Regarding Sharing

The monitoring user's terminal 3 may be set up to record (chronological) information/data regarding the eyes of the monitoring user themselves as well as those of the monitored user, a family member, for sharing as-necessary. An embodiment of this is described below.

(1) For example, data for contact lens information of the monitored user (brand, number, curve) is acquired by photographing using a camera or by linking data with a medical institution, and this can be stored in the area for dedicated applications in storage 13 by the monitoring user's terminal 3, in storage 23 by storing means 267 of the computer system 2, or in a storing region of the cloud (not shown).

Therefore, the monitoring user can place an order for contact lenses on behalf of the monitored user using the monitoring user's terminal 3. Also, having the monitoring user's terminal 3 share contact lens information of the monitored user with a medical institution and pharmacy enables examinations and prescriptions to proceed smoothly. Note, any orders, tests, or prescriptions requested in proxy by this monitoring user are, for example, preferably within the scope of existing prescriptions or diagnostic results for the monitored user.

(2) For example, clinical record information for the monitored user's eyes (for example, history of endothelial cells, visual acuity, refractive power, axial length, cataracts, glaucoma, or the like) can be acquired through data linkage with a medical institution and this can be stored in dedicated application area in storage 13 by the monitoring user's terminal 3, in storage 23 by storing means 267 of the computer system 2, or in a storage region of the cloud (not shown). Thus, the monitoring user can track health conditions of the eyes of the monitored user and easily notice any abnormalities. In addition, when the monitoring user operates the monitoring user's terminal 3 to share clinical record information of the monitored user's eyes with a medical institution or pharmacy, examinations and prescriptions can proceed smoothly.

(3) For example, the date the monitored user starts use of contact lenses, the date the monitored user purchases contact lenses, the date that the monitored user is to throw away the contact lenses, and eye examination date for the monitored user may be stored through manual input or input using a dedicated device using the monitoring user's terminal 3 or monitored user's terminal 1 to a dedicated application area in storage 13 by the monitoring user's terminal 3, in storage 23 by the storing means 267 of the computer system 2, or in a storage region of the cloud (not shown). Thus, the date the monitored user is to start using contact lenses, the date the monitored user purchases contact lenses, the date for a family member to throw away the contact lenses, and eye examination dates for the monitored user can be known, enabling taking actions to maintain healthy eyes.

For example, when inventory of contact lenses for the monitored user (for example, family of the monitoring user) is less than a set amount, the notifying means 263 can provide notification of an alert to the monitoring user (for example, to the monitoring user's terminal 3). In addition, if the monitored user (for example, family) is using contact lenses that have been used for one day or more or when it is time to replace contact lenses, the notifying means 263 may provide notification of an alert to the monitoring user (for example, to the monitoring user's terminal 3). In addition, if difficulty seeing using the contact lenses is detected (such as, for example, squinting more than normal) for the monitored user (for example, family), the notifying means 263 may provide notification to the monitored user (or monitored user's terminal 1) and/or the monitoring user (or monitoring user's terminal 3) proposing prescription strength replacement and/or a visit to an ophthalmologist. For example, if the monitored user (for example, family) is in their teens or twenties, the notifying means 263 may provide notification to the monitored user (or monitored user's terminal 1) and/or the monitoring user (or monitoring user's terminal 3) proposing colored contact lenses.

(4) For example, the [name of the] primary care doctor of the monitored user (for example, family of the monitoring user) can be acquired through manual input, photographing the patient registration card, or attaching web information and this can be stored in dedicated application area of storage 13 by the monitoring user's terminal 3, in storage 23 by the storing means 267 of the computer system 2, or in a storage region of the cloud (not shown). Also, registration is feasible for each of the monitored users. Therefore, the primary care doctor can be contacted immediately using the dedicated application installed on the monitoring user's terminal 3 if there are any abnormalities. In addition, future hospital appointments and past hospital visit history for the monitored user can be output to a calendar.

### SPECIFIC EXAMPLE (1)

### Step 1

After the monitored user receives an eye test or medical examination, (1) the results (information such as visual acuity, intraocular pressure, fundus of the eye, refractive power, axial length, cataracts, or glaucoma status) or primary care doctor/examination registration ticket information is acquired in paper form or on digital media.

### Step 2

The information acquired from the monitored user is shared with the monitoring user. For example, this authorization may include the regulatory required consent to share personal information such as diagnostic results.

### Examples of Sharing Methods

Dedicated application on the monitored user's terminal 1, dedicated application on the monitoring user's terminal 3: notification of diagnostic information associated with the monitored user's account can be provided to a monitoring user previously agreed to.
Medical institution—dedicated application on monitoring user's terminal 3: medical institution can provide notification of information online to the applicable monitored user or monitoring user.
On paper—dedicated application on monitoring user's terminal 3: information acquired on paper by the monitored user can be input to the system by hand or photographed and uploaded or the like enabling the monitoring user to receive notification of uploaded information.

### Step 3

The following is an example of what a monitoring user can do.
Information, reports, and service information provided by a medical institution or the like regarding information acquired for the monitored user can be received by the monitored user and monitoring user at the same time. Note, this doesn't have to necessarily be at the same time, where initially the monitored user is notified and if the monitored user does not take action (visiting a hospital, scheduling an exam, or the like) within a prescribed amount of time, the monitoring user is contacted.
The notifying means 263 can provide notification to not only the monitored user but also the monitoring user of a reminder to return to the hospital, or recommend additional tests.
The notifying means 263 can provide notification to the monitoring user of recommendations of products and services and the like matching the eye condition.
When the monitored user complains of physical problems or upon visiting a medical institution, the monitoring user can provide information regarding the monitored user to the medical institution facilitating appointments, tests, and prescriptions.

### SPECIFIC EXAMPLE (2)

### Step 1

The results of an eye exam or health checkup of the monitored user, a contact lens prescription, or prescription strength can be acquire on paper or digital medium.

### Step 2

The acquired information of the monitored user may be shared with the monitoring user (family (parents or children of senior citizens) or approved relatives). An example of a sharing method may be the same as specific example (1) described above.

### Step 3

The following is an example of what a monitoring user can do.
With prior consent of the monitored user or confirmation of a doctor, contact lenses can be purchased on behalf of the monitored user.

### SPECIFIC EXAMPLE (3)

### Step 1

Contact lens purchase history or purchase request history of the monitored user can be acquired in paper or digital medium.

### Step 2

The acquired information of the monitored user may be shared with the monitoring user (family (parents or children of senior citizens) or approved relatives). An example of a sharing method may be the same as specific example (1) described above.

### Step 3

The following is an example of what a monitoring user can do.
Notification of an alert indicating the date for the next purchase of contact lenses calculated based on the number of contact lenses purchased can be provided simultaneously or non-simultaneously to the monitored user and the monitoring user. Here, the monitoring user can be enabled to request purchase on behalf of the monitored user if the monitored user forgets to make the purchase. Thus, the inconvenience of forgetting to purchase contact lenses can be resolved.
Both the monitored user and the monitoring user may view purchase history of the other and notification may be provided as well. This prevents duplicate purchasing of contact lenses where both the monitored user and the monitoring user each purchase contact lenses.

### Embodiment of Monitoring and Alerting of Eye Condition of Family

As described above, the computer system 2 may monitor eye conditions of family who is the monitored user and provide notification of an alert to the monitoring user (or monitoring user's terminal 3). An embodiment of this is described below.

(1) If the monitored user is a senior citizen living separately from the monitoring user (for example, a parent of the monitoring user), it is difficult for the monitoring user to appropriately understand cataracts or progression of the monitored user. While the monitored user is using the monitored user's terminal 1, images/video of the monitored user's eyes can be acquired or interaction data can be acquired by photographing using the camera 17 of the monitored user's terminal. The judging means 264 of the computer system 2 judges severity based on a prescribed judgment algorithm and may issue an alert to the monitoring user (for example, child of monitored user who has reached adulthood) via the monitoring user's terminal 3.

(2) If the monitored user is a young person (for example, a child of the monitoring user), images/video of the monitored user's eyes can be captured using the camera 17 of the monitored user's terminal 1 while in use (for example, while playing a game) to learn about nearsightedness or farsightedness of the child, interaction data may be captured, or assessment data may be captured using a vision assessment tool in an application or installed in a pharmacy. The judging means 264 of the computer system 2 judges severity based on a prescribed judgment algorithm and may issue an alert to the monitoring user (for example, guardian or the like of the monitored user) via the monitoring user's terminal 3.

(3) Also, to maintain the mental health of the monitored user, behavior while performing work where the eyes are used (sometimes combined with weather information or behavior information other than for eyes such as location information) may be monitored and acquired. The judging means 264 of the computer system 2 judges severity related to mental health based on a prescribed judgment algorithm and may issue an alert to the monitoring user (for example, guardian or the like of the monitored user) via the monitoring user's terminal 3.

### Specific Example (1): Case Where Monitored User is a Child

### Step 1

Eye conditions/information of the monitored user (child) may be stored in storage 23 by the storing means 267 of the computer system 2 (for example, a server).

Example of information to be stored in storage 23 of computer system 2:
Photographs of the eyes captured by the camera 17 of the monitored user's terminal 1, another camera, or an automatic camera
Distance information between the monitored user's terminal 1 and the eyes
Use of smartphones, games, and TVs (time spent in contact with digital devices, contact conditions (continuous use or not), and the like)
Location information obtained from the monitored user's terminal 1 (for example, kid's cell phone)
Data from measurements using a dedicated application installed on the monitored user's terminal 1 or a vision assessment tool installed at a pharmacy and the like
Response results by the monitored user (child) for a “self-check test” (for example, questionnaire or simple eye exam) that the monitoring user (for example, parent) sends to the monitored user (child) at arbitrary timing

Information about the monitored user to be stored in storage 23 of the computer system 2 may be determined based on the attributes of the monitored user.

User attributes change with the passage of time and life events (growing up, moving, marriage, and the like), and the health information to be monitored will change accordingly. For this reason, the updating means 268 may update the user attributes and update the type of health information monitored according to updated user attributes.

When family of the monitored user (for example, a parent) or approved relative are registered as a monitoring user, notification of items to be monitored based on attributes (age, location, eye condition, life events (for example, going to school, marriage) and the like) is provided to the monitoring user. This is because it is likely that the age of the monitoring user is different from that of the monitored user and therefore the monitoring user may not have enough experience or information regarding health issues and not know what types of data need to be collected or what information will be delivered based on an alert.

### Step 2

The judging means 264 of the computer system 2 may issue an alert based on a judgment algorithm if a prescribed threshold is exceeded. For example, the notifying means may issue an alert in the following cases.

(Example 1) Case that squinting is confirmed using the camera 17 of the monitored user's terminal 1 for a predetermined amount of time

(Example 2) Case that digital device contact time exceeds a healthy upper limit (also called healthy contact time)

(Example 3) Case where it is estimated from location information of the monitored user's terminal 1 and weather information that the monitored user has spent a prescribed amount of time at a location where pollen, PM2.5, or ultraviolet light is higher than the standard

Note, the upper limit of contact with a digital device that is healthy is personalized and may be set based on eye conditions of the monitored user and/or the monitored user's age and/or external factors (for example, weather, pollen dispersal, amount of UV radiation, season, and the like).

Note, the alert threshold determining means 266 does not provide a uniform “digital device contact time threshold” for everyone. The digital device contact time threshold is personalized and set individually. For example, younger children have a contact time threshold set lower, days with high pollen count have threshold set lower, and various combinations of these.

The notifying means 263 may provide information regarding contact lens products or ophthalmic correction devices according to amount of pollen (or forecast value) or amount of UV radiation (or forecast value) to the monitored user (or monitored user's terminal 1) and/or the monitoring user (or monitoring user's terminal 3).

Specifically, for example, if the amount of dispersed pollen (or forecast value) is more than standard, the notifying means 263 may provide notification to the monitored user (or monitored user's terminal 1) and/or the monitoring user (or monitoring user's terminal 3) of a proposal to wear anti-pollen contact lenses.
Specifically, for example, if the amount of UV radiation (or forecast value) is more than standard, the notifying means 263 may provide notification to the monitored user (or monitored user's terminal 1) and/or the monitoring user (or monitoring user's terminal 3) of a proposal to wear contact lenses with UV protection.

### Step 3

Notification of alerts can be provided to the monitored user and the monitoring user (family of monitored user (parent) or approved relative) at the same time or only to the monitoring user. During an alert, notification of the following information may be provided.

(Example 1) Squinting was observed for 30 minutes. Consult with a medical institution.

(Example 2) Find a relaxing place to rest our eyes.
If this is set up in advance, the primary care doctor can also be informed when an alert is issued.

Note, the notifying means 263 can also provide notification of actions that should be taken in the event of an alert simultaneously to the monitored user and the monitoring user (family of monitored user (parent) or approved relative) or only to the monitoring user. This is because it is likely that the age of the monitoring user is different from that of the monitored user and that the monitoring user will have insufficient experience and information regarding health issues and may not have knowledge of what actions are necessary.

Also, for the case of issuing an alert, setting up a comparison of the general statistical data regarding issuing an alert (for example, average value of other registered users) and monitored user data is feasible. Knowing whether or not alerts are issued a lot or rarely for applicable persons helps in providing a sense of urgency regarding the alerts.

It is possible to enable confirming alert issuance history in chronological order for each user (each member of the family). Furthermore, the notifying means 263 can issue an additional alert if, in referencing the chronological data, an alert has been issued continuously for a certain period or issuance frequency has been high for a certain period of time.

The computer system 2 or administrator thereof may be set up to take some type of action (such as contact from the medical institution or automatically setting a medical appointment).

### Step 4

Information to submit to a medical institution (including chronological information) may be prepared for a person to seek medical attention from a medical institution based on an alert. For example, daily monitoring information can be edited and output in a prescribed electronic clinical record format making it easier to submit information to a doctor.

### Specific Example (2): Case Monitored User is a Senior Citizen

### Step 1

Eye conditions/information of the monitored user (senior citizen) may be stored in storage 23 by the storing means 267 of the computer system 2 (for example, a server).

Examples of information stored on the server:
Photographs of the eyes captured by the camera 17 of the monitored user's terminal 1, another camera, or an automatic camera
Distance information between the monitored user's terminal 1 and the eyes
Use of smartphones and TVs (time spent in contact with digital devices, contact conditions (continuous use or not), and the like)
Location information obtained from the monitored user's terminal 1
Data from measurements using a dedicated application installed on the monitored user's terminal 1 or a vision assessment tool installed at a pharmacy and the like
Response results by the monitored user for a “self-check test” (for example, questionnaire or simple eye exam) that the monitoring user (for example, child) sends to the monitored user at arbitrary timing

Information for the monitored user to be stored in storage 23 of the computer system 2 (for example, a server) may be determined based on the attributes of the monitored user.

User attributes change with the passage of time and life events (growing up, aging, moving, marriage, and the like), and the information to be monitored will change accordingly. For this reason, the updating means 268 may update the user attributes and update the type of health information monitored according to updated user attributes.

When family of the monitored user (parent) or approved relative are registered as a monitoring user, notification of items to be monitored based on attributes (age, location, eye condition, and the like) is provided to the monitoring user. This is because it is likely that the age of the monitoring user is different from that of the monitored user and therefore the monitoring user may not have enough experience or information regarding health issues and not know what types of data need to be collected or what information will be delivered based on an alert.

### Step 2

The judging means 264 of the computer system 2 may issue an alert based on a judgment algorithm on the server if a prescribed threshold is exceeded. For example, the notifying means may issue an alert in the following cases.

(Example 1) Case that cataract condition is confirmed using the camera 17 of the monitored user's terminal 1

(Example 2) Case where due to eye conditions of the monitored user and/or monitored user's age and/or external factors (for example, weather, amount of pollen dispersed, amount of UV radiation, season, or the like), care is needed for going out as visual acuity outdoors will be lower than usual

Note, the alert threshold determining means 266 does not provide a uniform “threshold of caution for going out” but personalizes and sets the threshold individually based on age, location of the monitored user, and sunshine intensity. For example, in the case of a senior citizen with a tendency toward cataracts, the sunlight intensity threshold may be set lower or the threshold may be set lower for days with high pollen counts or various combinations of these.

### Pattern 2

Sending information to the monitoring user regularly (in other words, a set time such as 9:00 every morning) is feasible. This can be used in the watching over and monitoring of health of a parent living separately. If monitored information is sent in real time, the monitoring user (child) can monitor activities of the monitored user (senior citizen). For example, monitoring daily activities is feasible.

In addition, the judging means 264 of the computer system 2 may judge based on a judgment algorithm and the notifying means 263 may issue an alert that a certain threshold has been exceeded.

(Example 1) The monitored user (senior citizen) can take a picture of their own eyes using the camera 17 (or a smartphone equipped with an external device for testing) of the monitored user's terminal 1 and send this to the computer system 2. With confirmation provided because a picture is sent every day, the monitoring user (child) is able to check up on the living conditions of the monitored user (senior citizen). On the other hand, artificial intelligence (AI) of the computer system 2 may judge the risk level of cataracts and may be set up so that the notifying means 263 issues an alert if the risk level is high.

(Example 2) The monitored user (senior citizen) can take a picture of their own eyes using the camera 17 (or a smartphone equipped with an external device for testing) of the monitored user's terminal 1 and send this to the computer system 2. On the other hand, step count data acquired from a pedometer or pedometer application of the monitored user's terminal 1 or location data acquired from the monitored user's terminal 1 is sent to the computer system 2. Even if risk of cataracts is moderate, the notifying means 263 may issue an alert in the case that there are several days where number of steps data is too low or location information indicates being near motionless for several days.

### Step 3

An alert can be provided to the monitored user and the monitoring user (family of monitored user (child of senior citizen) or approved relative) at the same time or only to the monitoring user. During an alert, notification of the following information may be provided.

(Example 1) There is a tendency for cataracts. Consult with a medical institution.

(Example 2) The weather is poor today and sunshine intensity seems to be weak. Be careful when going out.
If this is set up in advance, the primary care doctor can also be informed when an alert is issued.

Note, the notifying means 263 can also provide notification of actions that should be taken in the event of an alert simultaneously to the monitored user and the monitoring user (family of monitored user (parent) or approved relative) or only to the monitoring user. This is because it is likely that the age of the monitoring user is different from that of the monitored user and that the monitoring user will have insufficient experience and information regarding health issues and may not have knowledge of what actions are necessary.

In the case of issuing an alert, setting up a comparison of the general statistical data regarding issuing an alert (for example, average value of other registered users) and monitored user data is feasible. Knowing whether or not alerts are issued a lot or rarely for applicable persons helps in providing a sense of urgency regarding the alerts.

It is possible to enable confirming alert issuance history in chronological order for each user (each member of the family). Furthermore, the notifying means 263 can issue an additional alert if, in referencing the chronological data, an alert has been issued continuously for a certain period or issuance frequency has been high for a certain period of time.

The computer system 2 or administrator thereof may be set up to take some type of action (such as contact from the medical institution or automatically setting a medical appointment).

### Step 4

Information to submit to a medical institution (including chronological information) may be prepared for a person to seek medical attention from a medical institution based on an alert. For example, daily monitoring information can be edited and output in a prescribed electronic clinical record format making it easier to submit information to a doctor.

Note, display style can be adjusted automatically when the monitoring user shares information with the family. For example, when sharing content with the family, the method of expressing the content may be changed based on the person shared with. For example, this can be changed to content for children (for example, content such as picture books) if shared with children. For the elderly, the text can be made larger and fewer colors used.

Note, at least a part of the computer system 2 described in the embodiment above can be configured using hardware or configured using software. If configured using software, the program for expressing at least a part of the functionality of the computer system 2 may be stored to storage media such as a flexible disk, CD-ROM, or the like and read into a computer for execution. The storage media is not limited to removable types such as magnetic disks or optical disks and can be fixed storage media such as a hard disk device or memory or the like.

In addition, programs expressing at least a part of the functionality of the computer system 2 can be distributed via a communication line such as the internet (including wireless communication). Furthermore, the program may be distributed through a wired line or wireless circuit such as the Internet or by being stored in the recording medium in an encrypted, modulated, or compressed state.

Furthermore, the computer system 2 functionality can be provided by one or a plurality of information devices. In a case where a plurality of information devices are used, one may be a computer, and such computer may execute a predetermined program to achieve a function as at least one means of the computer system 2.

Furthermore, in the invention of the method, all processes (steps) may be realized in automatic control by a computer. Moreover, the progress control between the processes may be performed by the human hand while each process is being performed by the computer. In addition, at least some of all the processes may be performed by human hand.

Therefore, the present invention is not limited to the embodiments described above, and the components can be modified and embodied within a range not deviating from the gist of the present invention at the implementing stage. Various inventions can be formed by appropriate combinations of a plurality of components disclosed in the embodiments described above. For example, several components may be deleted from all the components shown in the embodiments. Furthermore, the components across different embodiments may be appropriately combined.

### EXPLANATION OF CODES

1 Monitored user's terminal
11 Input interface
12 Communication module
13 Storage
14 Memory
15 Display
16 Processor
161 Receiving part
162 Processing part
163 Communication controller
17 Camera
2 Computer system
21 Input interface
22 Communication module
23 Storage
24 memory
25 Output interface
26 Processor
261 Determining means
262 Acquiring means
263 Notifying means
264 Judging means
265 Proxy contacting means
266 Alert threshold determining means
267 Storing means
268 Updating means
269 Outputting means
3 User terminal
4-1 to 4-N Medical institution terminals
S information processing system

1. An information processing system, comprising:
determining means for determining the type of monitored information to monitor for a monitored user according to user attributes of the monitored user;
acquiring means for acquiring monitored information of the monitored user corresponding to the determined type; and
notifying means to provide notification of acquired monitored user monitored information and/or information for monitoring health according to the determined type to the monitoring user associated with the monitored user.

2. The information processing system according to claim 1, wherein
the notifying means provides notification of the determined type to the monitored user and requests this type of information of the monitored user and
the acquiring means acquires the information sent according to this request as monitored information.

3. An information processing system, comprising:
acquiring means for acquiring monitored information of the monitored user;
judging means for judging a health condition of the monitored user using the acquired monitored information of the monitored user based on judgment criteria; and
notifying means to provide notification of acquired monitored user monitored information and/or information for monitoring health for the determined type based on the judgment results to the monitoring user associated with the monitored user.

4. The information processing system according to claim 3 further comprising:
determining means for determining which type of monitored information to monitor for the monitored user according to user attributes of the monitored user, wherein
the acquiring means acquires monitored information of the monitored user corresponding to the determined type.

5. The information processing system according to claim 3 or 4, wherein the judging means determines judgment criteria according to user attributes of the monitored user.

6. The information processing system according to any one of claims 3 to 5, wherein the notifying means provides notification of at least one of:
information of the acquired monitored information of the monitored user formatted to enable submitting to a medical institution;
information enabling the monitoring user to communicate with a medical institution on behalf of the monitored user; or
information related to a diagnosis or treatment regarding health condition judged to be poor, abnormal, or to have deteriorated based on judgment criteria or to the medical institution,
as information including acquired monitored information of the monitored user and/or information for monitoring health of the determined type.

7. The information processing system according to any one of claims 3 to 6, wherein the acquiring means acquires at least one of images and video of the eyes of the monitored user, eye movements obtained from the images and video of the eyes, and interaction data indicating behavior of work where eyes are used as monitored information, and the judging means judges the health condition of the eyes of the monitored user.

8. The information processing system according to any one of claims 3 to 7 further comprising:
an alert threshold determining means for determining time and/or frequency of viewing a digital device screen that is healthy according to user attributes of the monitored user and/or external factors, as an alert threshold, wherein
the acquiring means acquires time and/or frequency of viewing a digital device screen by the monitored user as monitored information, and
the judging means judges whether or not the time and/or frequency of the monitored user viewing a digital device screen exceeds the threshold.

9. The information processing system according to any one of claims 3 to 8, wherein user attributes are information expressing individual attributes of the monitored user and the monitoring user that is information related to health and living environment including age, sex, vision, presence or absence of vision correction, chronic illness, medical history, surgical history, allergies, place of residence, living environment, living with other family members, and lifestyle.

10. The information processing system according to any one of claims 3 to 9, wherein the notifying means provides notification of information including acquired monitored information of the monitored user and/or information for monitoring health of the determined type, or information including information related to user attributes of the monitored user or information related to user attributes of the monitoring user.

11. An information processing method, comprising:
a determination step of determining the type of monitored information to monitor for a monitored user according to user attributes of the monitored user;
an acquiring step for acquiring monitored information of the monitored user corresponding to the determined type; and
a notifying step to provide notification of acquired monitored user monitored information and/or information for monitoring health according to the determined type to the monitoring user associated with the monitored user.

12. An information processing method, comprising:
an acquiring step for acquiring monitored information of the monitored user,
a judging step for judging a health condition of the monitored user using the acquired monitored information of the monitored user based on judgment criteria; and
a notifying step to provide notification of acquired monitored user monitored information and/or information for monitoring health for the determined type based on the judgment results to the monitoring user associated with the monitored user.
